(19) United States
(12) Patent Application Publication
(10) Pub. No.: US 2011/0054370 A1
(43) Pub. Date: Mar. 3, 2011

(54) Knee joint separation brace

(76) Inventor: Lloyd George Perry, Fort Wayne, IN (US)

(21) Appl. No.: 12/806,989

(22) Filed: Aug. 25, 2010

Related U.S. Application Data
(60) Provisional application No. 61/275,163, filed on Aug. 26, 2009.

(51) Int. Cl. A61F 5/00

(52) U.S. Cl. 602/16

(57) ABSTRACT

This brace supplies a helping hand to those persons that for one reason or another can not have knee surgery. Bone infections, allergic reaction to materials, even cost they can't afford all prevent the repair. The intent here is to do something no other brace does, it separates the joint just slightly so that the bone on bone does not rub each other and so gives relief from swelling and pain. The mechanism that does all the work without putting pins in bones for traction, is versitile. Used on an operating table the modified cam can assist in anything from back surgery to joint and bone surgery. The advantage of traction is there but without the use of the drilled and pinned bones. This makes for quick prep and shorter recovery time.

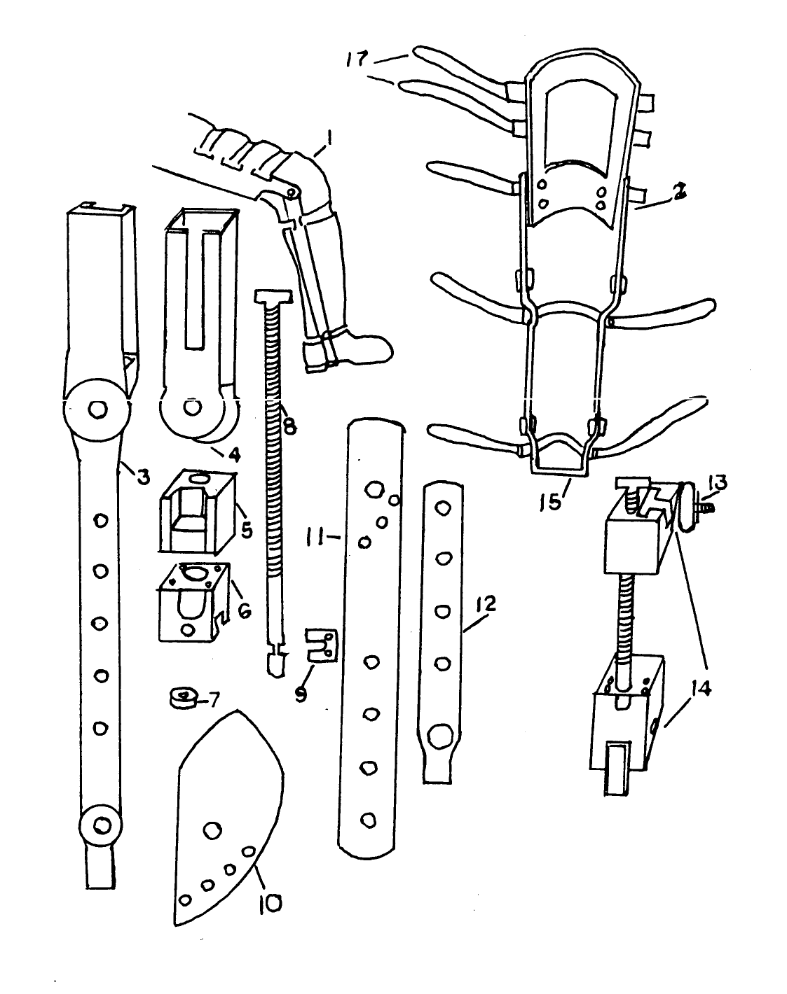

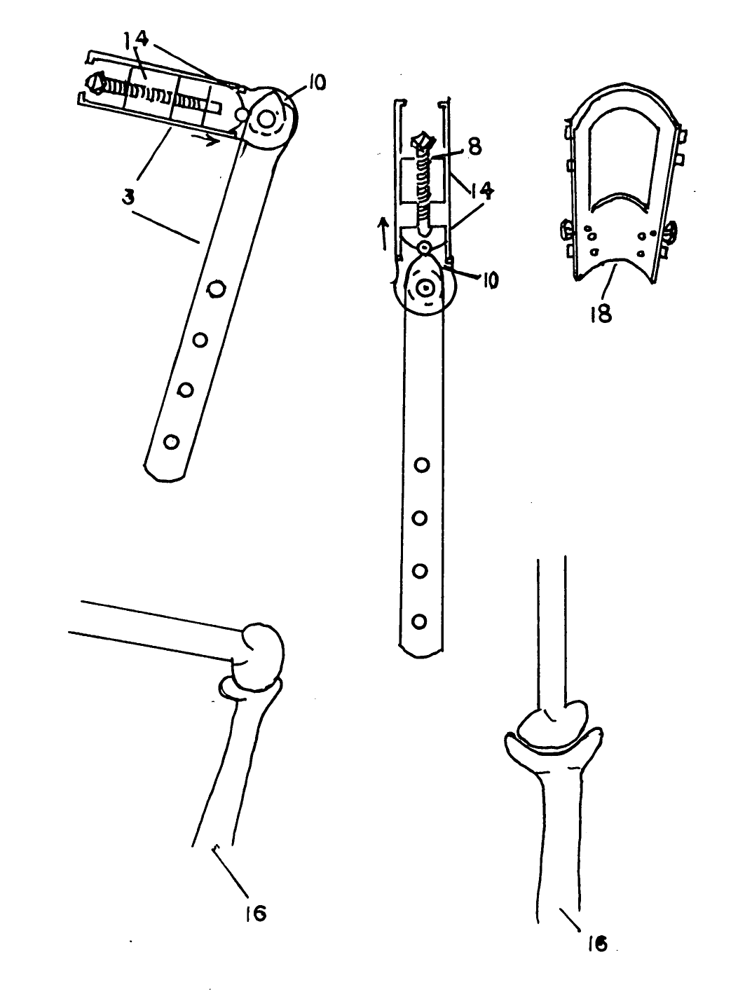

This nonprovisional U.S. utility patent is a dependent claim establish by a provisional U.S. patent filing Aug. 26, 2009 under U.S. No. 61/275,163, by Lloyd G. Perry, a U.S. Citizen, residing in the State of Indiana, County of Allen, in the city of Fort Wayne.

### BACKGROUND OF THE INVENTION

No government funds nor assistance was used to develop this invention.

In research for prior patents, many braces were found, none fit this discription.

This brace is intended for use in the separation of the knee joint for patients that have worn out joints but can not have a knee replacement. Such conditions have the joint parts wearing bone on bone with the absence of cartilage. Conditions such as infection of the bone; diabetes; allergies to the materials used in the repair or replacement of the joint are some of the reasons people can't have knee replacement or knee repair using any foreign material. Thus the need for this type of brace to gain relief and regain mobility.

The fact that the knee joint is rubbing bone on bone makes for continued inflammation of the joint, reducing range of motion and even causing the joint to lock in a fixed position. The primary function of this brace is to separate that bone on bone condition by slightly pushing the upper and lower parts of the joint apart just a few thousands of an inch so that thay no longer rub bone on bone. Inflammation is reduced, swelling goes down and mobility returns.

In past treatments the separation process ment tapping a pin into the bone of the upper and lower sections of the leg and forcing the separation of the knee joint with screws and turn-buckles placed between those said pins. This was not desired for long term use and could not be used in some circumstances for the same reasons as with the knee replacement. The brace eliminates both the risks and the undesired procedure giving relief where relief could not otherwise be obtained.

### BRIEF SUMMARY OF THE INVENTION

This invention was brought about by the personal needs of this inventor. Having spent 5 months in the hospital with a staff infection in the bone of the left leg, both wrists and back bone, caused by flea bites, made mobility next to impossible.

The request for some sort of device to aid in the use of the leg was answered with, “we don't have anything that will separate the bones of the knee and you can not have a replacement because of the risk of a return of the infection.”

Mechanical engineering is in my background and so the concept was worked out. Now some time later, the needs of others are being expressed, so the knee joint separator will be made available for all to use. It's functions are not limited to just the separation of the joint but also to bone alignment made possible by the adjustable cams that will allow the pitching of the alignment toward the corrective position. Attachments can also be fastened to the brace to help that bone alignment condition hold it's corrected position.

### BRIEF DESCRIPTION OF THE SEVERAL VIEWS OF THE DRAWINGS AND IDENTIFICATION OF PARTS

1. Shows the brace installed on the leg.

2. Shows the full brace not installed.

3. Full length assembly of one side of the brace.

4. Housing for the slide mechanism containing 5,6,7,8,9 as an assembly

5. This is both a seat for the frame to attach to this block by use of 13, (see 18) a guide for the slide assembly and a thread base for the long threaded slack adjuster screw 8.

6. This is the bottom slide guide that carries the ball bearing cam roller 7. and is the anchor for slack adjuster 8. held in place by clip 9.

7. Ball bearing cam roller mounts in bottom of 6, pinned in place.

8. Threaded slack adjuster to take up the slack to insure that the cam causes the separation of the knee joint.

9. Clip to hold the slack adjuster rod in place in the bottom slide guide.

10. Adjustable cam that mounts as an assembly with 11. within the pivit joint of 4.

11. Adjustable bar that attaches to the cam on one end and the lower section of the brace number 3. with bar number 12 forming the length from the top joint that joins 3 and 4 going down to the stirrup 15. forming one full side of the brace with a like assembly on the other side of the brace. However both cams and sides do not have to be adjusted the same if there is a need to pitch the bones in a corrective direction. Adjusting the cams can make that correction.

12. Connecting bar between 11. and the ankle joint that connects the stirrup 15 to the assembly.

13. This is a button type attachment to the frame of the brace and that rides in the pocket of part #5, allowing the adjustment of the slack out, between the upper section of the brace and the cam followers, insuring separation with the cam movement.

14. The complete slack adjuster and slide guide assembly that fits into 4.

15. Stirrup with holding straps.

16. Bare bone joints showing the bone on bone compared to the separation joint.

17. There are 5 sets of straps made with vel cro to easy fasten the brace in place.

18. Top section before connecting with lower section by means of button attachment that couples to the slide assembly mounted to the full length brace. This section is strapped on the leg and then the button installed in the slide assembly seat.

### DETAILED DESCRIPTION OF THE INVENTION WITH FUNCTION OF THE BRACE

1. Using the illistration parts identification guide and the parts identification list; you see that the full and complete brace is intended to be placed under the foot and strapped fast and extend all the way up to the very top of the brace as is illistrated in #2 illistration.

2. The purpose of the brace is to cause the upper section of the knee joint and the lower section of the knee joint to separate just a little bit (10 thousands of one inch) so as to take the direct rubb of bone on bone out of the joint movement. This reduces inflammation and pain when no other method of physical change can be used.

3. To see how this is accomplished, we look at the full length brace in illistration #3 and see that it appears to be one solid bar from stirrup to top of brace. Well it is. However in the top section of the #3 assembly (marked #4) is a hollow slide assembly pictured in illistration #14 as an assembly. Also shown as parts numbered 4,5,6,7,8,9,10. And as a separate two part piece in #18.

4. On page 2 of 2 of the drawings you see illistration #3 again but in an open view showing you that the slide assembly #14 slides toward the center pivit point of the brace (knee) when the leg is bent. This is relieving the pressure and the knee joint closes as is shown in the left hand side of illistration #16 and below it. This is accomplished by the cam (illistration #10) moving away from the cam follower (ball bearing) mounted on the ends of the #14 assembly.

5. The illistration to the right of the bent brace, is an illistration of the brace and leg streight. In this illistration you see the cam #10 pointing streight into the cam follower (ball bearing follower) with the slide mechanism going away from the center pivit point of the brace (knee) the illistration #16 of the bare bone joint (on the right) show the joint separated just slightly.

6. The push that is put on the upper and lower sections of the knee joint is caused by the cam rolling up on the ball bearing followers and forcing the slide assembly to move upward.

7. Illistration #13 is a metal button or wheel that rides inside the slide assembly #14 in a special pocket for it to set in. (shown in illistration #5 and 18) That button (located on both sides of the brace) attaches to the upper part of the brace, causing the upper part of the brace to push the theigh from the knee up. The lower part of the leg is held in place by the stirrup and strapps at the calf of the leg and around the ankle and arch of the foot. Thus the weight of the person wearing the brace holds the lower section down so that the spread can occure.

8. For the brace to be effective it must first be properly fitted to the person wearing it. Thus, make sure, by measuring the leg circumference, that there is a funnel type taper made in the upper part of the brace between the knee and the thigh. By keeping that natural tapper in the brace, the meat of the leg will tend to seat it's self in that taper, thus helping to prevent slippage of the brace when the push of the cam is heavy during separation. Adjust the brace pivit points for the knee and ankle to match the joints pivit points.

9. So that proper clearances can occure in the knee joint when the leg is streightened, the slack or the natural movement of the skin on the upper part of the leg must be adjusted out. That is accomplished by turning the long threaded screw (illistration #8) with the adjustment tool or screwdriver to take the slack out of that skin movement. To do this, mount the upper part of the brace first. Tight but not to tight. Follow this by mounting the lower part of the brace, making sure that the button (illistration #13 and 18) is in it's seat. Tighten your straps for the lower part of the brace. Once they are tight, bend the leg as shown in illistration #1. Tighten the long screw to the point that it extends the ball bearing through the housing for the slide assembly. Gently streighten the leg and test to see if sufficient pressure will be applied by the cam to cause separation. If not, tighten a little more and repeat the process until the pressure is there. Keep in mind that your leg is not use to this, so go slow each time you put it on and don't try to get to much to fast. Stretching those joint parts will be hard to do because they are made to hold the joint together. Keep in mind that you are only after a few thousands of an inch. (the thickness of a piece of stationary paper) As you get use to the brace you will learn short-cuts in putting it on and you may find that you need to adjust it after you have had it on for a while.

10. In aid of other problems with the knee and/or ankle, positioning attachments can be put on the frame of the brace along with the adjustment of varied cam adjustments, all of which can help with leg alignment for corrective bone alignment caused by degenerative conditions. The cams do not have to be set in the same place on both sides of the leg brace, if there is a need to vary that adjustment so as to pitch the bones in a corrective direction the cam adjustments can do this.

11. There is nothing hard about making this brace other than the bending of the metal and drilling of the holes. To look at the drawings, the building and assembly is self exclamatory. The only needed information not noted in the drawings are the particular specifications suited for each person the brace is made for. The girth of the leg from just above the knee (2″) up toward the hip, approximately 7 inches (varies with the persons height) must be measured so as to be able to build the brace with the required funnel shape taper.

12. Vel cro straps should be used so as to be able to tighten and adjust the brace with ease. In adjusting the brace be sure to make the pivit point of the brace at the same pivit point of the joint. (knee or ankle)

1. The uniqueness of this brace is in the mechanism with the cam assembly that leverages the separation of the joint it is applied to.
DEPENDENT CLAIM ∩1. The cam mechanism of claim #1 of this brace can be modified and installed for use in any application where traction is required.
DEPENDENT CLAIM ∩2. The cam mechanism of claim #1 can be modified and installed on operating table to assist in surgery where traction devices would be needed.
DEPENDENT CLAIM ∩3. The cam mechanism of claim #1 can be used in smaller applications such as finger splints to set and hold a bone or joint in place.
DEPENDENT CLAIM ∩4. The design and structure of the brace in claim #1 can carry other corrective devises by mounting them on the brace.
DEPENDENT CLAIM ∩5. The brace in claim #1 can be modified to be fully adjustable in both height and girth settings if there would be a need for the versitility.
